(19) United States
(12) Patent Application Publication
(10) Pub. No.: US 2008/0288528 A1
(43) Pub. Date: Nov. 20, 2008

(54) Systems and methods for detecting toxins in a sample

(76) Inventor: Scott Gallager, North Falmouth, MA (US)

Correspondence Address: ROPES & GRAY LLP, PATENT DOCKETING 39/41, ONE INTERNATIONAL PLACE, BOSTON, MA, 02110-2624, US

(21) Appl. No.: 11/804,462

(22) Filed: May 18, 2007

(51) Int. Cl. G06F 17/30; C12Q 1/02

(52) U.S. Cl. 707/102; 435/29; 707/E17.055

(57) ABSTRACT

In one aspect the invention provides a system for detecting the presence of toxins in a sample that includes a plurality of chambers for culturing organisms and observing the organism's motility response when introduced into a sample containing a toxin. The toxicity measurement system may include an imaging module to monitor and track the movement of one or more organisms in the sample and identify abnormalities. In other aspects, the invention provides methods of culturing organisms and detecting the presence of toxins in the sample using the motility response of organisms in the sample.

### BACKGROUND

Contamination by toxic industrial chemicals can pose a threat to civilian and military drinking water supplies. There is also growing concern over terrorist and natural disaster caused contamination of drinking water supplies. Yet, there is a lack of commercial technology capable of efficiently and reliably testing for non-specific toxins in real time.

Current methods utilize studying the growth rate and cell size of microorganisms such as protozoa. Previous studies have indicated that the growth rate and cell size of such microorganisms are modified in the presence of a variety of toxic substances. However, such measurement can take long periods of time to complete. Furthermore, such tests require fairly sophisticated and consequently expensive equipment.

Other methods consist of studying the movement patterns of cells in the presence of toxic substances. However, these methods merely study bulk statistics of the movement behavior and determine if there are any abnormalities. These methods therefore lack sophistication and the ability to measure the degree and extent of contamination.

Accordingly, there is a need for a simple, economical system that has sufficient sophistication to study the toxicity of samples.

### SUMMARY OF THE INVENTION

The invention provides for systems and methods having more degrees of specificity than merely a threshold for abnormality, because some substances are not very toxic in small quantities and are even required for the ecology. Moreover, the systems and methods described herein provide a more holistic method, incorporating information about cell growth, movement and cell size among other things to study the toxicity of samples. In general, the systems and methods described herein include improved systems and methods to identify the presence of a toxin in a sample using an organism's motility response when introduced into a sample containing the toxin.

In one aspect, the invention provides a system for detecting the presence of toxins in a sample that includes a plurality of chambers for culturing organisms and observing the organism's motility response when introduced into a sample containing a toxin. The toxicity measurement system may include an imaging module to monitor and track the movement of one or more organisms in the sample and identify abnormalities. In other aspects, the invention provides methods of culturing organisms and detecting the presence of toxins in the sample using the motility response of organisms in the sample.

More particularly, in one aspect, the systems and methods described herein include an apparatus to identify the presence of a toxin in a sample. The apparatus comprises a first chamber, a second chamber, an imaging module and a processing module. The first chamber contains an organism having a motility response to a toxin in a sample. The first chamber may include at least one of small volume tubes, IV (Intravenous) bags, spinner flasks, and plastic bags. The second chamber may have a fluid connection with the first chamber, and may include an opening such that the sample may be introduced through the opening and combined with the organism from the first chamber. The second chamber may include a transparent glass enclosure. The imaging module may be electromagnetically coupled to the second chamber and capable of imaging a path of the organism in the second chamber. The processing module may be connected to the imaging module such that the imaged path is processed to detect the presence of a toxin in the sample.

The apparatus may further comprise a sampling pump and a valve for regulating the movement of the organism and sample into the second chamber. The sampling pump and valve may include a syringe type pump and a valve system. In certain embodiments, the sampling pump and valve comprises at least one supply unit in fluid connection with the sampling pump and valve for supplying at least one of acid, bleach, water and the sample.

In certain embodiments, the organism include at least one of a single-celled organism, multi-celled organism, fresh water organism, and salt water organism. The organism may include at least one of a ciliate, flagellate and marine algae. The ciliate may include at least one of Tetrahymena pyriformis, Tetrahymena malaccensus, Tetrahymena furgisoni, and Glaucoma. The flagellate may include at least one of Tetramitus and Bodo, and the marine algae may include at least one of Chlorophycia, rhodomonas, heterocapsa, dunaliella, and chlamydomonas.

In certain embodiments, the sample includes a fluid and the sample includes water obtained from at least one of public water supply, reservoirs, taps, treatments plants, military portable water tanks, and field installations. In such embodiments, the toxin includes at least one of heavy metals, cellular respiration inhibitors, cellular energy transfer disruptors, carcinogens, acetylcholinesterase inhibitors, reactive oxygen species, general oxidants, substances influencing sugar metabolism, and water disinfection products. The toxin may also include at least one of cadmium chloride, potassium cyanide, sodium azide, ethylene glycol, sodium arsenate, Alflatoxin B, parathion, paraquat, methane methyl sulfonate (MMS), hydrogen peroxide, methyl nitrosoamine, and chlorine.

In another aspect, the invention provides a method of detecting the presence of a toxin in a sample. The method may comprise the steps of providing a chamber including a sample and an organism having the motility response to a toxin in the sample, and capturing an image of a portion of the chamber having the organism in the sample. The method may further comprise detecting the presence of a toxin in a sample by monitoring the motility response of the organism in the sample, and measuring the change in the modulated response. The motility response of the organism is monitored by tracking the path of the organism at it moves in sample.

In one embodiment, the step of detecting the presence of a toxin may comprise further the steps of acquiring an image showing an organism in a sample at a first instance in time, and measuring based on the image at least one of the centroid, the size of the organism, the shape of the organism, and orientation of the organism. The method may further include the steps of determining a path characteristic based at least on the image and a similar image from another instance in time, and detecting the presence of a toxin in a sample based at least on the path characteristic. In certain embodiments, at least one of the steps of capturing an image and acquiring an image includes obtaining an electronic image using a camera.

The path characteristic may include at least one of speed, acceleration, direction displacement, and rate of change of direction. In one embodiment, the step of determining a path characteristic may include estimating a path characteristic based on a mathematical model of the movement of the organism and the captured image. In such embodiments, mathematical model comprises dynamical system models including Kalman filter based model. In another embodiment, the step of monitoring the motility response and measuring a change in the motility response includes measuring a net to gross displacement ratio of the path characteristic. In such an embodiment the step of detecting the presence of a toxin includes determining if the net to gross displacement ratio is above or below a threshold. The motility response may include at least one of speed, acceleration, direction, displacement and rate of change of direction. The step of determining a path characteristic includes determining a path of an organism using at least one of a nearest neighbor distance algorithm, voronoi tessellation algorithm and an autocorrelation algorithm.

In certain embodiments, the method comprises the step of building a database of toxins including providing a set of toxins, providing at least one organism having a motility response to at least one set of toxins and introducing the organism to the at least one of the set of toxins. The method may further comprise measuring the motility response of the at least one organism to the at least one of the set of toxins, and building a database having the at least one of the set of toxins, and corresponding motility response of the at least one organism. In such embodiments, the step of identifying a toxin in a sample includes providing a chamber including the sample and an organism having the motility response to a toxin in the sample, capturing an image of the portion of the chamber having the organism moving in the sample, and identifying a toxin in the sample by comparing the motility response of the organism to a motility response of an organism in a database of toxins.

In another aspect, the invention provides a method of detecting the presence of a toxin in a sample comprising the steps of culturing an organism having a motility response to a toxin in a first medium, and culturing the organism in a second medium, such that the second medium is removed and replenished at regular intervals of time. The method further includes the steps of introducing the cultured organism to a sample at regular intervals of time and detecting the presence of the toxin in the sample based at least on a motility response of the culture organism to the toxin. In one embodiment, the first medium may include at least one of bacteria based medium and a yeast based medium. The first medium and/or the second medium may include at least one of MS1 or yeast extract. The step of culturing the organism in the first medium may include performing the static culture and the step of culturing the organism in the second medium may include performing a continuous culture.

### BRIEF DESCRIPTION OF THE DRAWINGS

The following figures depict certain illustrative embodiments of the invention in which like reference numerals refer to like elements. These depicted embodiments may not be drawn to scale and are to be understood as illustrative of the invention and not as limiting in any way.

FIG. 1 is a conceptual block diagram depicting a system for detecting toxins in a sample according to one illustrative embodiment of the invention.

FIG. 2 is a more detailed block diagram depicting a system for detecting toxins in a sample according to one illustrative embodiment of the invention.

FIG. 3 is a flow diagram depicting a method detecting a toxin according to one embodiment of the invention.

FIG. 4 is a flow diagram depicting a method of controlling the operation of the system for detecting toxins in a sample according to one illustrative embodiment of the invention.

FIG. 5 is a flow diagram depicting a software subroutine for imaging a sample according to one illustrative embodiment of the invention.

FIG. 6 is a flow diagram depicting a software subroutine to track paths of the organism in the sample according to one illustrative embodiment of the invention.

FIG. 7 is a flow diagram depicting a software subroutine of FIG. 6 to track existing paths according to one illustrative embodiment of the invention.

FIG. 8 is a flow diagram depicting a software subroutine of FIG. 6 to track new paths according to one illustrative embodiment of the invention.

FIG. 9 is chart depicting tracking particles according to one illustrative embodiment of the invention.

FIG. 10 depicts a graphical user interface (GUI) according to one illustrative embodiment of the invention.

FIG. 11 depicts a scheme for building a database having organism responses to various toxins according to one illustrative embodiment of the invention.

### DETAILED DESCRIPTION OF THE ILLUSTRATED EMBODIMENTS

These and other aspects and embodiments of the systems and methods of the invention will be described more fully by referring to the figures provided.

The systems and methods described herein will now be described with reference to certain illustrative embodiments. However, the invention is not to be limited to these illustrated embodiments which are provided merely for the purpose of describing the systems and methods of the invention and are not to be understood as limiting in anyway.

As will be seen from the following description, in one aspect the invention provides a system for detecting the presence of toxins in a sample that includes a plurality of chambers for culturing organisms and observing the organism's motility response when introduced into a sample containing a toxin. The toxicity measurement system may include an imaging module to monitor and track the movement of one or more organisms in the sample and identify abnormalities. In other aspects, the invention provides methods of culturing organisms and detecting the presence of toxins in the sample using the motility response of organisms in the sample.

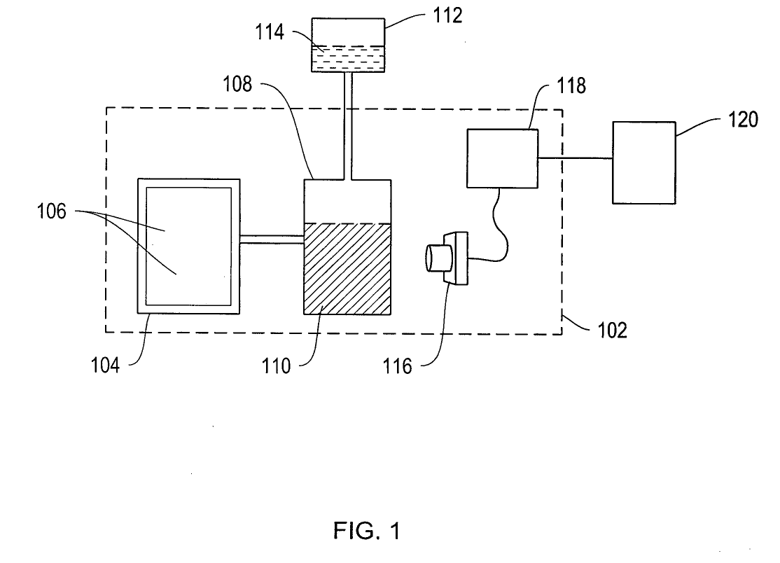

FIG. 1 is a conceptual block diagram depicting a system for detecting toxins in a sample according to one illustrative embodiment of the invention. In particular, FIG. 1 depicts a toxicity measurement system 102 including a culture chamber 104, an observation chamber 108, and imaging module 116 and a computer terminal 118. The observation chamber 108 includes an organism-sample mixture 110 comprising a sample 114 to be tested for toxicity and an organism 106 having a motility response to a toxin in the sample 114. The observation chamber 108 is in fluid connection with the culture chamber 104. The organism 106 is cultured in the culture chamber 104 and supplied to the observation chamber 108. The observation chamber 108 is also in fluid connection with a sample reservoir 112. A sample 114 is collected in a sample reservoir 112 and is supplied to the observation chamber 108.

The movement pattern of the motile organism 106 is affected by the presence of toxins in the sample 114. As an example, organisms 106 such as ciliated protozoa of the genus Tetrahymena, Glaucoma and Tetramitus show sensitivity to a variety of toxins such as heavy metals, organophosphates, disinfectants and other industrial chemicals. In such examples, the toxins inhibit or stimulate calcium transport across the organism's 106 ciliar membrane. Calcium is typically responsible for the depolarization of the ciliar membrane. A loss or excess of calcium may either slow down or excite the beat frequency of the cilia. Consequently, a change in beat pattern or frequency typically alters the rotational torque applied to a fluid (in this example, the fluid includes sample 114) by the organism 106. Such an alteration in the rotational torque may generally result in a more circuitous movement behavior. In one embodiment, the movement behavior of the organism 106 may be tracked in the sample 108 by the imaging module 116.

During operation of the toxicity measurement 102, the organisms 106 are cultured in the culture chamber 104 such that their growth rate may be monitored and/or controlled. At certain desired intervals of time, the organisms 106 are introduced into the observation chamber 108 to interact with the sample 114. The sample 114 is introduced into the observation chamber 108 from the sample reservoir 112. The organisms 106 may be chosen from a set of organisms that are motile in certain solutions. In one embodiment, the organism 106 tracks a substantially linear path through the mixture 110. However, in the presence of certain substances including toxins, the organism 106 tends to deviate from the substantially linear path. The path of the organism 106 and any deviations can be tracked using cameras and image processing algorithms capable of tracking the organism 106 as it moves in the mixture 110. The tracked behavior of the organism 106 is collected and processed in the computer terminal 118 and may be viewed in display 120.

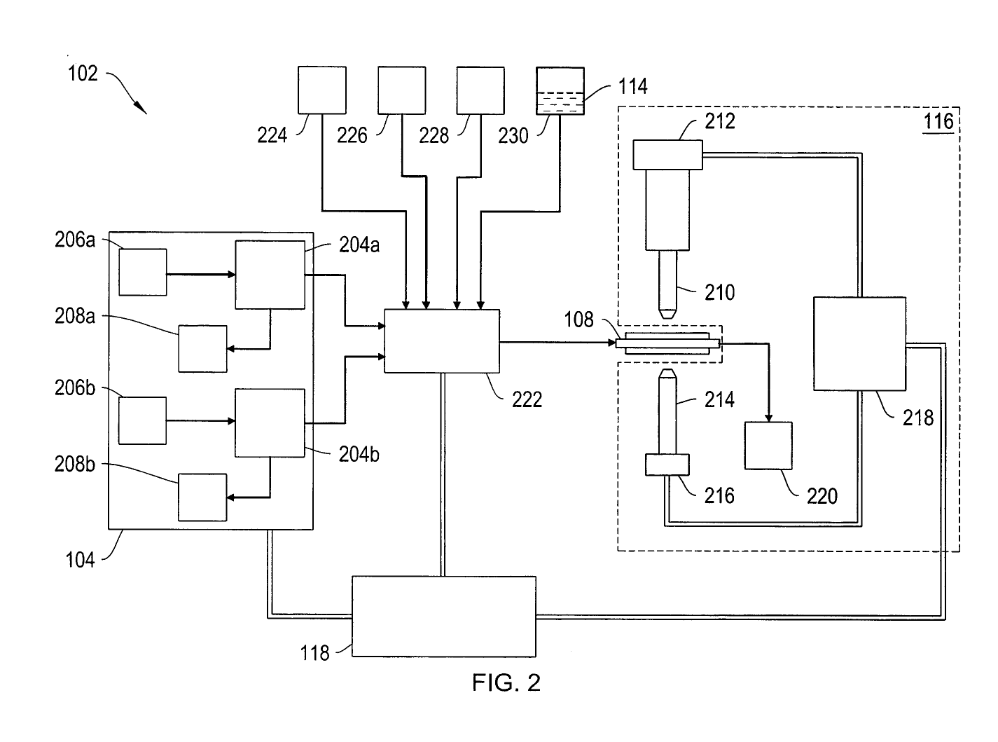

FIG. 2 is a more detailed block diagram depicting a system 102 for detecting toxins in a sample according to one illustrative embodiment of the invention. The toxicity measurement system 102 includes the culture chamber 104, the observation chamber 108, the imaging module 116 and the computer terminal 118. The toxicity measurement system 102 also includes a sampling valve 222 connected to the culture chamber 104 and the observation chamber 108. The sampling valve 222 is also connected to a water supply 224, a bleach supply 226, an acid supply 228 and a sample supply 230. The sample 114 from the sample supply 230 is combined with the organism 106 from the culture chamber 106 through sampling valve 222 and sent to the observation chamber 108 for analysis. The organism-sample mixture 110 can be removed from the observation chamber 108 and sent to a waste disposal unit 220.

In one embodiment, the culture chamber 104 includes bioreactors 204a and 204b (generally, “bioreactor 204”), media supply units 206a and 206b (generally, “media supply unit 206”) and waste disposal units 208a and 208b (generally, “waste disposal unit 208”). During operation, media from the media supply unit 206 may be introduced into the bioreactor 204 and combined with an organism 106. The media may optionally be removed from the bioreactor 204 and sent to the waste disposal unit 208. The media in combination with suitable environmental conditions may promote the growth of the organism 106 being cultured. The cultured organism 106 may be supplied to the observation chamber 108 through the sampling valve 222 for imaging. In certain embodiments, the culture chamber 104 may include one or more bioreactor valves to regulate the flow of media into the bioreactor 204 and the flow of waste out of the bioreactor 204.

The bioreactor 204 may be suitably designed to operate in static culture schemes and in continuous culture schemes. In static culture schemes, the organism 106 may be combined with a culture medium and allowed to grow and populate the bioreactor 204. In the static culture scheme, the medium may generally be kept unchanged. In continuous culture schemes, the medium may be constantly replenished by regularly adding new quantities of media from the media supply unit 206 and removing, from the culture, used media into the waste disposal unit 208. In some embodiments, the bioreactor 204 may be separate from media supply units 206 and waste disposal units 208. In such embodiments, the separated bioreactor 204 may be included in the culture chamber 104 in addition to a bioreactor 204 connected to the media supply units 206 and waste disposal units 208.

The bioreactor 204 includes small-volume tubes including polycarbonate tubes, glass flasks, spinner flasks, plastic bags. The culture chamber 104 may include one or more bioreactors 204, each may have the same or different organism 106 being cultured. In certain embodiments, the bioreactor 204 includes culture flasks having a volume of about 100 mL, and the media supply unit 206 includes IV bags, having a capacity of about 500 mL, that are connected to the culture flasks by a syringe pump. In such embodiments, the turnover rate of the media into and out of the bioreactor 204 may be about 1/day giving an organism volume of 100 mL per day at an organism concentration of about 104 organisms/mL.

The organism 106 may include single celled and/or multi-celled organisms. The organism 106 may include fresh water and salt water organisms. The organism 106 may include flagellates and/or ciliates. In certain embodiments, the organism 106 includes marine algae. The flagellates may be selected from a group comprising of Tetramitus and Bodo. The ciliates may be selected from a group comprising Tetrahymena pyriformis, Tetrahymena malaccensus, Tetrahymena furgisoni, and Glaucoma. The marine algae may be selected from a group comprising of Chlorophycia, rhodomonas, heterocapsa, dunaliella, and chlamydomonas.

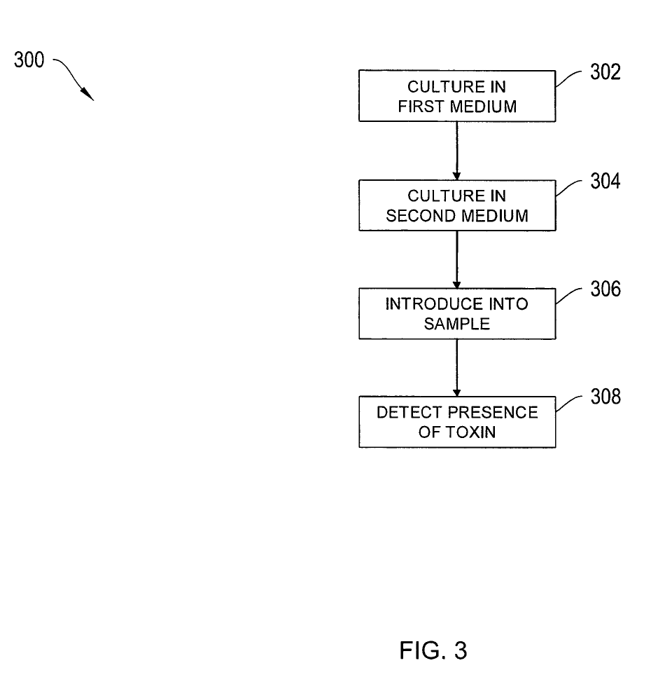

In certain embodiments, the organism 106 is cultured in a first medium and then cultured in a second medium before combining with a sample 114. In such embodiments, the first culture may be a static culture and the second culture may be a continuous culture. FIG. 3 is a flow diagram depicting such a method 300 to detect the presence of a toxin in the sample. In particular, the organism is cultured in a first medium (step 302). In one embodiment, this step 302 is typically a static culture scheme. In such an embodiment, organisms 106 such as protozoa may be combined with a bacteria-based medium in small-volume tubes to perform a static culture. In one example, the organism 106 includes cultures of Glaucoma chattoni that may be obtained from American Type Culture Collection (ATCC), Manassas, Va. and kept at 20 C until introduction into a medium. The static culture may be maintained in polycarbonate tubes containing about 10 percent Klebsiella pneumoniae suspension in MS1 medium. Klebsiella pneumoniae may be obtained from Environmental Toxicity Laboratory (ETL). MS1 medium for tetrahymena typically includes proteose-peptone medium, tryptone, K 2HPO4, and distilled water. One or more static cultures may be maintained to grow a population of the organism 106. In certain embodiments, about 2 mL cultures are cultured at 20 C on a water-bath shaker table, while about 5 mL cultures are kept unshaken in an adjacent room as back-ups and seed stock for the continuous cultures.

The organism 106 is then cultured in a second medium (step 304). In one embodiment, the step 304 is typically a continuous culture scheme. In such an embodiment, the static cultures are combined with a second medium such as a yeast-based medium. In particular, the second medium may be stored in flasks and may include about 100 mL of about 1% yeast extract media in distilled water. The yeast extract media typically includes about 5 g of yeast extract (Sigma Y-1626) and about 100 mL MS1 media.

The static cultures from step 302 are combined with the second media in spinner flasks (about 125 mL). These spinner flasks are typically used as a bioreactor 204 for continuous culture schemes. In one embodiment, the spinner flasks typically receive about 110 mL of the second media (about 1% yeast in MS1 media) daily. In one embodiment, a similar amount of the second media is dispensed out of the bioreactor 204 into the waste disposal unit 208. In such an embodiment, the turnover rate of the second media into and out of the bioreactor 204 is about 0.9 per day. The temperature during continuous culture is typically set to about 27 C+/−2 C. In certain embodiments, as noted earlier, parallel cultures may be kept at a cooler temperature of about 20 C in a suitable culture facility.

The cultures are introduced into the sample 114 (step 306) and the presence of a toxin in the sample 114 may be detected based, at least in part, on the movement of the organisms 106 in the sample 114. The sampling and imaging of the organism-sample mixture 110 is explained in more detail below with reference to the elements of the toxicity measurement system 102 of FIGS. 1 and 2.

Returning to FIG. 2, the organism 106 may be provided from the culture in bioreactor 204. In certain embodiments, culture chamber 104 includes a plurality of bioreactors 204, each providing one or more species of organisms 106. The organism 106 may be combined with the sample 114 from sample supply unit 230.

In one embodiment, the sample supply unit 230 may be a continuous sample source such that desired doses of the sample 114 may be supplied to the observation chamber 108 on a regular basis. In such an embodiment, the sample supply unit 230 is connected to at least one of public water supply, reservoirs, taps, treatment plants, military portable water tanks and field installations. In certain embodiments, the sample supply unit 230 is a static sample source having about 500 μL of the sample 114. The sample supply unit 230 is capable of providing a sample 114 having toxins.

In one embodiment, the toxins include at least one of heavy metals, cellular respiration inhibitors, cellular energy transfer disruptors, carcinogens, acetylcholinesterase inhibitors, reactive oxygen species, general oxidants, substances influencing sugar metabolism, and water disinfection products. The toxins may include at least one of cadmium chloride, potassium cyanide, sodium azide, ethylene glycol, sodium arsenate, Alflatoxin B, parathion, paraquat, methane methyl sulfonate (MMS), hydrogen peroxide, methyl nitrosoamine, and chlorine. The toxins may include other substances capable of stimulating a motility response in an organism 106 without departing from the scope of the invention. Concentration of toxins in the sample may range from trace quantities to about 10 mg/mL. The concentration of toxins in the sample being tested may vary depending on the nature of the toxin and the organism 106. In certain embodiments, the concentration of toxins range from trace quantities to about 50 ug/mL. The sample supply unit 230 is connected to the observation chamber 108 through sampling pump/valve 222.

The sampling pump/valve 222 is connected to sample supply unit 230 and serves to pump the sample 114 and the organism 106 into the observation chamber 108 for imaging and toxicity measurement. The sampling pump/valve 222 may include a syringe type pump and valve system. The sampling pump/valve 222 is connected to the culture chamber 104 and more particularly to one or more bioreactors 204.

In one embodiment, the observation chamber 108 may need cleaning due to dead organisms and other debris clinging to its insides. In such embodiments, the observation chamber is cleaned with cleaning agents such as water, bleach, and acid. Consequently, the sampling pump/valve 222 is also connected to an acid supply 228, a bleach supply 226 and a water supply 224. During operation, the sampling pump/valve 222 may pump water, bleach and acid from the corresponding supply units 224, 226 and 228, respectively into the observation chamber 108. The sampling pump/valve 222 may also be operated in regular manner such that the cleaning and imaging cycles may be conducted in a predetermined schedule. In certain embodiments, the sampling pump/valve 222 pumps at least the sample 114 and organism 106 at time intervals of about 1 to about 10 minutes, depending, among other things, on the toxicity of the sample 114.

The observation chamber 108 may be formed from clear, transparent materials such as glass or plastic. In one embodiment, the observation chamber 108 includes filters to selectively allow light of one or more wavelengths to pass. In certain embodiments, the observation chamber 108 is aligned with the imaging module 116 such that the sample-organism mixture 110 can be imaged.

The imaging module 116 includes an objective 210 and a camera 212 connected to an image processing engine 218. The imaging module 116 also includes a dark field condenser 214 and a strobe 216 connected to the image processing engine 218. The image processing engine 218 is connected to a computer terminal 118. During operation, light from the strobe 216 may impinge on the organism-sample mixture 110 in the observation chamber 108 through the dark field condenser 214. Reflected and/or refracted light from the sample-organism mixture 110 may be passed through into the microscope objective 210 and captured digitally by the camera 212. The image so obtained may be sent to the image processing engine 218 for further analysis and display.

In certain embodiments, the strobe 216 includes a pulsed light source. In other embodiments, the strobe 216 includes an arc lamp, an incandescent bulb which also may be colored, filtered or painted, a lens end bulb, a line light, a halogen lamp, a light emitting diode (LED), a chip from an LED, a neon bulb, a fluorescent tube, a fiber optic light pipe transmitting from a remote source, a laser or laser diode, or any other suitable light source. Additionally, the strobe 216 may be a multiple colored LED, or a combination of multiple colored radiation sources in order to provide a desired colored or white light output distribution. For example, a plurality of colored lights such as LEDs of different colors (red, blue, green) or a single LED with multiple colored chips may be employed to create white light or any other colored light output distribution by varying the intensities of each individual colored light. The strobe 216 may include a ring of LEDs to generate a circular source of light.

The dark field condenser 214 may include optical elements capable of directing light from the strobe 216 at oblique angles towards the observation chamber 108. In certain embodiments, the dark field condenser 214 directs light such that a hollow cone of illumination is produced that is focused on the sample-organism mixture 110. The light on the sample-organism mixture 110 in the observation chamber 108 is at an oblique angle to the surface of the observation chamber 108. The oblique light comes to focus on the sample-organism mixture 110 and then diverges such that light is prevented from entering the objective 210. However, light that is reflected or refracted by the sample-organism mixture 110 may pass through to the objective 210.

The light passing through the objective is collected by the camera 212. Camera 212 includes Charge-Coupled Devices (CCD) video sensor chip. The CCD converts the image into an electrical signal and sends it to an image processing engine 218 where it can be processed.

The image processing engine 218 may include a microprocessor or microcontroller that is programmed to process digital information from the CCD camera video sensor chip. The image processing engine 218 includes software algorithms for performing other functions such as identification of the organism 106 in the mixture 110, tracking the organism 106 and maintaining path information. The path information of the organism 106 may be sent to the computer terminal 118 for further processing and extracting statistics.

The computer terminal 118 may include any computer system having a microprocessor, a memory and a microcontroller. The memory typically includes a main memory and a read only memory. The memory may also include mass storage components having, for example, various disk drives, tape drives, etc. The mass storage may include one or more magnetic disk or tape drives or optical disk drives, for storing data and instructions for use by the microprocessor. The memory may also include one or more drives for various portable media, such as a floppy disk, a compact disc read only memory (CD-ROM), or an integrated circuit non-volatile memory adapter (i.e. PC-MCIA adapter) to input and output data and code to and from microprocessor. The memory may also include dynamic random access memory (DRAM) and high-speed cache memory.

In one embodiment, the computer terminal 118 is connected to the culture chamber 104, the sample valve 222 and the image processing engine 218. The computer terminal 118 controls the operation of the culture chamber 104. In particular, the computer terminal 118 monitors and regulates the flow of media and waste into and out of the bioreactor 204. In certain embodiments, the computer terminal 118 may also control the operation of bioreactor valves that may subsequently regulate the movement of media and waste to and from the bioreactor 204.

In one embodiment, the computer terminal 118 controls the operation of the sampling valve 222 such that organisms 106 from the culture chamber 104 is combined with water, bleach, acid and portions of the sample 114. In particular, computer algorithms may be implemented to control at least one of the timing and quantity of each of the elements in the observation mixture.

The computer terminal 118 may control the operation of the image processing engine 218. In certain embodiments, the computer terminal 118 may also control the operation of other elements in the imaging module 116 including the objective 210, the camera 212, the dark field condenser 214 and the strobe 216. The computer terminal 118 may also be used to control the position of the observation chamber 108.

In one embodiment, the computer terminal 118 includes a central processing unit (CPU), a communication/Ethernet module, a digital input/output module, a data acquisition module and serial interface module. In addition, the computer Terminal 118 may include relays, motion controls and one or more power supplies. The computer terminal 118 may include other external and internal modules that can operate with the toxicity measurement system 102. These modules may include a data logging module, an imaging module, a heating/cooling module, sensors, valves and pumps.

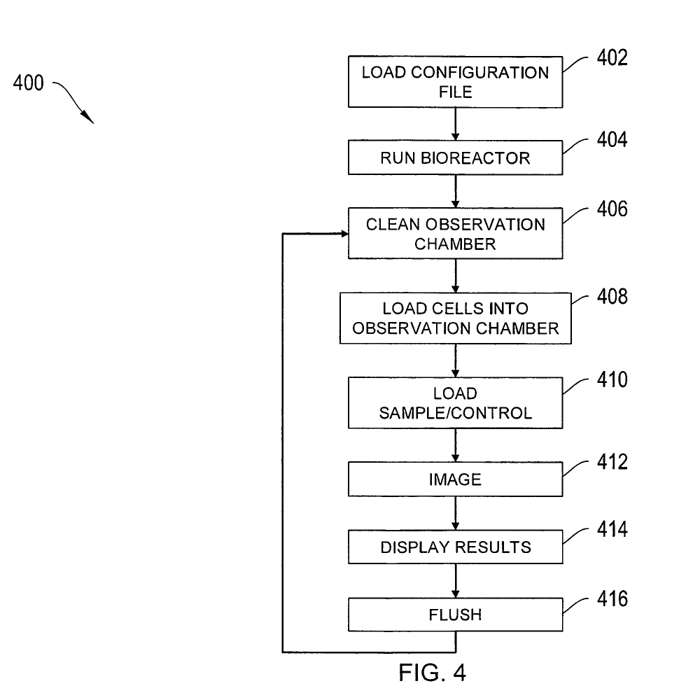

The operation of the toxicity measurement system 102 is described more fully with reference to FIG. 4 and subsequent figures. FIG. 4 is a flow diagram depicting a method 400 of controlling the operation of the system for detecting toxins in a sample according to one illustrative embodiment of the invention. In particular, FIG. 4 shows a method 400 of automatically controlling the various stages of testing a sample 114 for the presence of toxins. In one implementation the method 400 is implemented in software and run through the computer terminal 118 such that the operation of each element of the toxicity measurement system 102 is individually controlled. The method 400 begins with loading a configuration file (step 402) having values for configuration parameters and test conditions. The configuration file may include information about the nature of the test being conducted, the duration of operation of the bioreactor, bioreactor valves, sampling pump/valves and imaging system. The configuration file may also include values for environmental test conditions that can be controlled such as temperature, pressure and humidity. The bioreactor 204 in the culture chamber 104 is operated (step 404) to culture the organisms 106.

The observation chamber 108 is cleaned (step 406) prior to loading the organisms 106 into the observation chamber 108. In one embodiment, the observation chamber 108 may be cleaned by flushing it with bleach and then a surfactant and finally distilled water. The organism 106 is loaded into the observation chamber 108. The sample 114 is also loaded into the observation chamber 108 (step 410). In certain embodiments, a control sample of distilled water is added in the observation chamber 108 to study the movement patterns of the organisms 106 in non-toxic water. The observation chamber 108 with the sample 114 and the organisms 106 is then imaged (step 412) using the imaging module 116 and the results are processed by the computer terminal 118 and displayed in the display 120 (step 414). The results of the toxicity measurement including a measured alarm level indicating the level of toxicity in the sample 114 may be viewed on the display 120. The sample 114 and the organism 106 are then flushed out of the observation chamber (step 416) into the waste disposal unit 220.

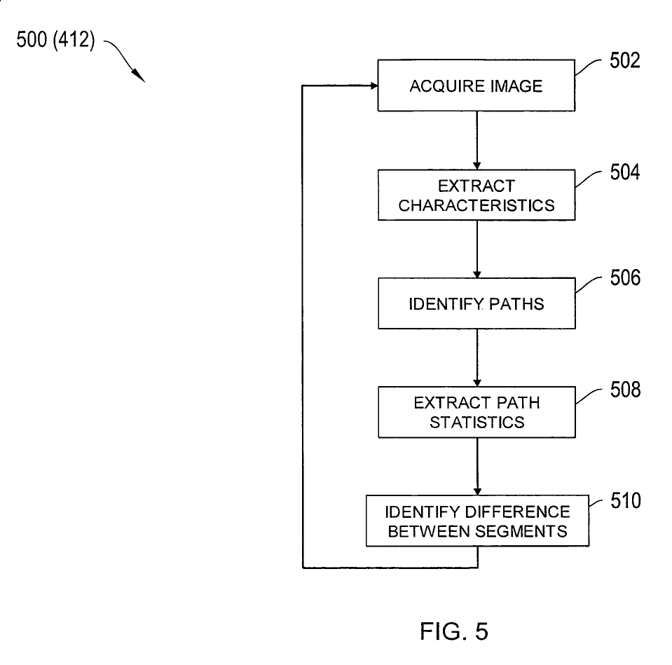

FIG. 5 is a flow diagram depicting a software subroutine 500 for imaging a sample according to one illustrative embodiment of the invention. In particular, the subroutine 500 corresponds to step 412 in FIG. 4 and begins by acquiring an image from the imaging module 116 (step 502) and extracting desired characteristics from the images (step 504). Organism 106 paths are then identified from the image (step 506). Certain path statistics are extracted from the image (step 508) and differences between the paths are calculated (step 510). The subroutine 500 returns to acquire a new image (step 502) in the next time step and iterates until the test has been deemed to be completed.

The image may be acquired (step 502) using the camera 212 of the imaging module 116. In one embodiment, the camera 212 captures an image of a portion of the observation chamber 108 containing the organism-sample mixture 110. The camera 212 may be configured to capture a series of images that may be combined to produce a video. In one embodiment, the camera 212 is configured to capture 30 images (frames) per second. The captured image is then sent to the image processing engine 218 for processing and analysis. The captured image may be divided into smaller regions based on the locations of desired path segments of the motile organism 106 or on particular regions of interest (ROI) determined earlier. Desired characteristics of the acquired images such as the location of the center of gravity of the organism (centroid), the size, shape, axes and orientation of the organism 106 may be extracted (step 504). In certain embodiments, the organism-sample mixture 110 may include a plurality of organisms 106. In such an embodiment, the desired characteristics from each of the plurality of organisms are extracted. In other embodiments, desired characteristics are extracted from a portion of organisms 106 in the organism-sample mixture 110.

The organisms 106 are distributed throughout the organism-sample mixture 110 and consequently appear as a set of shapes distributed throughout each acquired image. The determination of the centroid in step 504 may help simplify the representation of each organism 106 in the image from a complex shape to a single centroidal point. The acquired image may then comprise a two dimensional plot having one or more centroidal points at different locations on the diagram. The organisms 106 are typically motile and therefore keep moving from one location in the organism-sample mixture 110 to another. Each image may be acquired at a different instance in time and therefore, each acquired image may have a different distribution of centroidal points. This is because the organisms 106 being represented by the centroidal points may have moved to a different location in the organism-sample mixture 110. In one embodiment, the movement of the centroidal points (and therefore the organism 106) may be tracked and a path may be identified (step 506).

In step 506 two or more acquired images at different instances in time may be used to track the movement of centroidal points and thereby identify a path of an organism 106 in an organism-sample mixture 110. The centroidal points corresponding to one organism 106 from an image may be matched up with centroidal points corresponding to the same organism 106 from another image. In this way, a plurality of organisms 106 may be tracked simultaneously. A nearest neighbor distance algorithm may be implemented to compare the two or more images and determine possible paths for the one or more organisms 106 in the mixture 110. In the nearest neighbor distance algorithm, each centroidal point in an image is compared to the plurality of centroidal points from another image to determine a likely candidate for a matching centroidal point based on the closest distance between the centroidal points being compared. In other embodiments, the image comprising a plurality of centroidal points is divided into voronoi regions (polygonal regions having a centroidal point). The voronoi regions from one image may be compared and matched to the voronoi regions from another image and thereby matching the corresponding centroidal points and consequently the organism 106. In another embodiment, statistical techniques such as autocorrelation may be employed to match centroidal points and identify organism 106 paths. Other suitable techniques may also be employed to match centroidal points and identify the path of the organism 106 without departing from the scope of the invention.

In certain embodiments, other characteristics relating to the path and movement of the organism 106 can be extracted (step 508) from one or more images. In one embodiment, at least one of speed, acceleration, direction, rate of change of direction and distance may be extracted from the calculated path. The path characteristics may be stored in a data base in the computer terminal 118 from where statistics on a desired set of parameters may be extracted after a desired interval of time.

In one embodiment, an underlying dynamical system model may be used to model the movement of the organism 106 in the sample 114. In one example, the dynamical system model may be a Markov chain built on linear operators perturbed by Gaussian noise. The model may be built in accordance with the framework of a Kalman filter. A Kalman filter is a mathematical algorithm typically used in recursive estimation. Recursive estimation is a method for estimating the state of a system in the current step of recursion from the estimated state of the previous step and a measurement or observation from the current step. Kalman filter and the corresponding dynamical system model may be used to continuously update information about the position, velocity, size, orientation and brightness of the organism 106 being imaged. The Kalman filter-based model may be used to track and predict path characteristics for one or more organisms 106 simultaneously.

In certain embodiments, other characteristics such as statistical quantities are used to calculate the nature of the path being tracked. In one example, the net to gross displacement ratio (NGDR) is used to determine whether the paths being tracked are straight or circuitous. NGDR may range from about 0 to about 1, where values closer to 1 represent straighter paths than those represented by values closer to 0. In other embodiments, the deviation from an expected path may be used to determine the presence of a toxic substance in a sample 114. In certain embodiments, the path of the organism 106 in the sample 114 may be compared with the path of the organism 106 in another sample. In still other embodiments, the path may be divided into smaller segments and these segments are compared against one another. Such a comparison and analysis may be made using mathematical tools such as principal component analysis and support vector machines. In one embodiment, the path of the organism 106 in a sample 114 is compared with the path of the organism 106 in a control sample (e.g., distilled water).

As noted earlier, the image of the organism 106 in the mixture 110 is acquired using a camera 212. The camera typically captures a portion of the observation chamber that may define an observation window. Organisms 106 within the metes and the bounds of the observation window may be tracked by the imaging module 116. During imaging of the observation chamber 108, a plurality of organisms 106 may move in and out of the observation window. Therefore, in addition to tracking the paths of organisms already within the observation window, the imaging module 116 may identify and track new paths for organisms just entering the window.

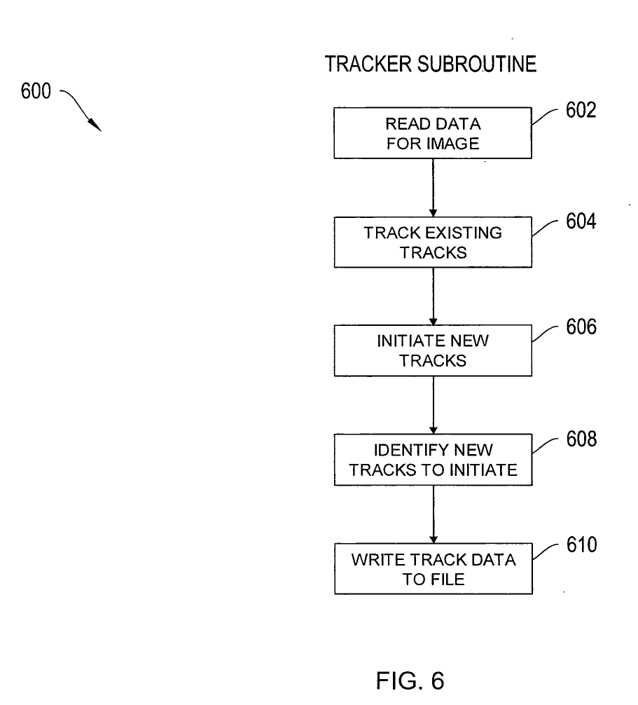

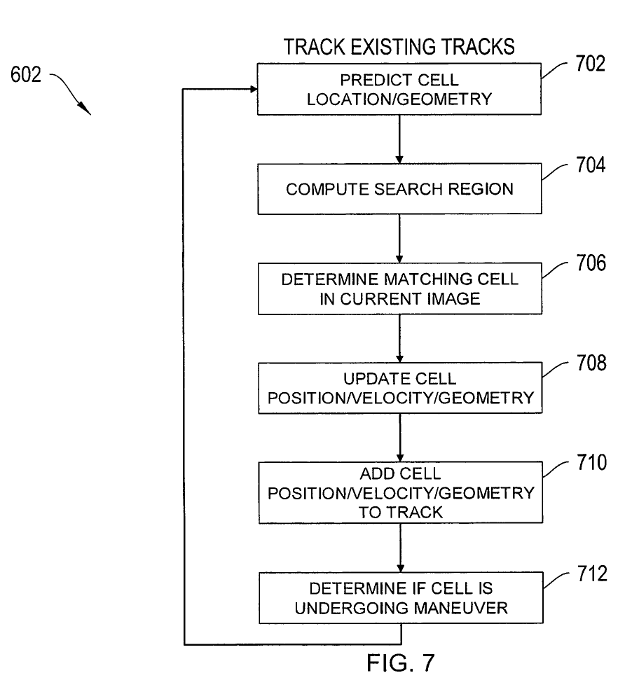

FIG. 6 is a flow diagram depicting a software subroutine 600 to track paths of the organism in the sample according to one illustrative embodiment of the invention. The subroutine 600 begins with reading the data and acquiring an image of an observation window (step 602). As noted earlier, the observation window is a portion of the observation chamber 108 that is being observed by the imaging module 116. The tracker subroutine 600 tracks existing paths for organisms 106 within the observation window (step 604). The subroutine 600 initiates new paths for organisms 106 that have just entered the observation window (step 606). The subroutine 600 also identifies new organisms 106 that are entering the observation window so as to prepare new paths for them (step 608). The subroutine 600 writes and saves the path data to file (step 610) within the computer terminal 118 to enable post-processing and analysis. The method 600 of FIG. 6 is described in more detail with reference to FIGS. 7, 8 and 9.

FIG. 7 is a flow diagram depicting a software subroutine 602 of FIG. 6 to track existing paths according to one illustrative embodiment of the invention. The subroutine 602 begins with the step of predicting the location and/or geometry of the organism 106 (702). Based, at least in part, on the organism's 106 location, a search region can be computed (704) and a matching organism 106 can be identified in the image being observed (706). The position and/or velocity and/or geometry of the organism is updated (708) and the new position and/or velocity and/or geometry is added to the tracked path (710). The subroutine 602 then determines if the organism 106 is undergoing a maneuver (712). The subroutine 602 is repeated until completion of the toxicity measurement. In one embodiment, NGDR calculation is used to determine if the organism 106 is undergoing a maneuver.

In one embodiment, the number of organisms 106 in the observation window may increase when new organisms 106 enter the window. In such an embodiment, the number of organisms 106 in the observation window becomes greater than the number of paths being tracked. The organisms 106 that have just entered the window may be identified. In one embodiment, once these organisms 106 are identified, their paths can be tracked.

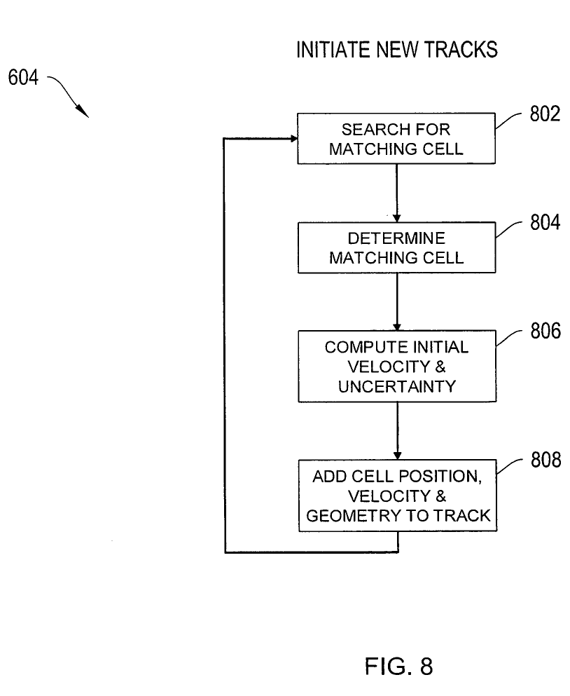

FIG. 8 is a flow diagram depicting a software subroutine 604 of FIG. 6 to track new paths according to one illustrative embodiment of the invention. New organisms identified in step 606 are now assigned paths in subroutine 604. The subroutine 602 begins with the step of searching for matching organisms 106 (step 802). Organisms 106 may be matched using a similar method as described in method 500 of FIG. 5. The matching organisms 106 are typically the identified 804 and their initial velocity and uncertainty is computed 806. In addition, the position and/or velocity and/or geometry of the organism 106 are included to the path 808.

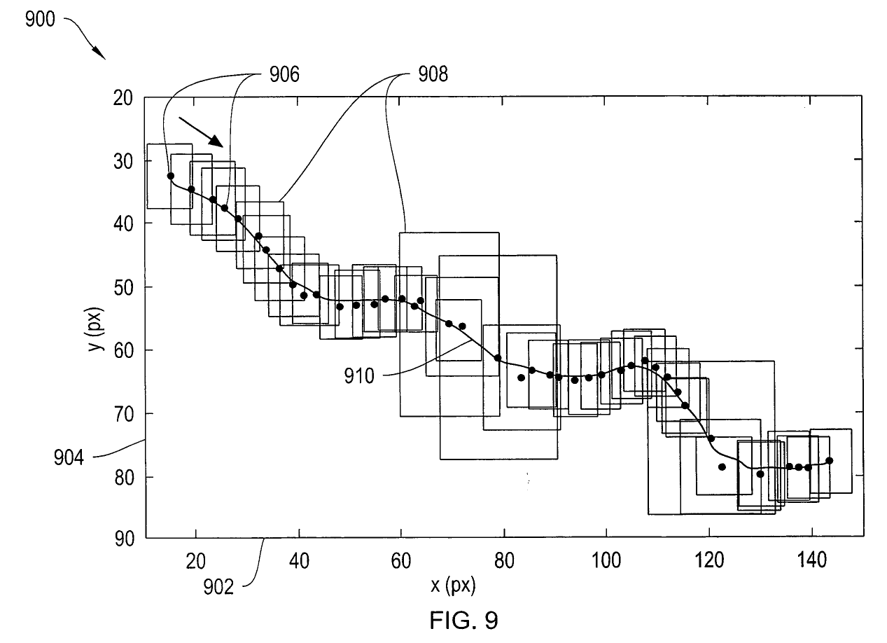

FIG. 9 is chart 900 depicting tracking particles according to one illustrative embodiment of the invention. Chart 900 shows the movement of the organism along the horizontal direction 902 and the vertical direction 904. Since, chart 900 is a computerized image of the location of the organism from image processing steps shown in the previous figures, the horizontal 902 and vertical 904 directions are measured in pixels. Chart 900 is a composite image of the organism shown as particles 906 at various locations in the observation window (e.g., the observation window in FIG. 9 is the boundary of the chart 900). Boxes 908 show uncertainty in the location of the particles 906 with larger boxes indicating higher uncertainty in prediction. The boxes 908, thereby, also depict a region within which the subroutines 500 and 600 can perform a search to find a matching organism 106. The movement of the organism 106 is tracked and a path 910 can be traced through the centers of the boxes 908.

Methods 500 and 600 identify and track each of a plurality of organisms 106 in the observation window. Graph 900 displays the path of an organism 106 being tracked during a toxicity measurement test. In addition to tracking the organism 106 and tracing its path in the mixture 110, statistical calculations may also be made to estimate the performance of the toxicity measurement test and plot consolidated results of the toxicity of the sample 114. In certain embodiments, image processing techniques are applied to the electronically captured image of the sample to remove or minimize the effects of undesirable artifacts such as external debris that may have collected in the sample. Such calculations and techniques may be performed using software algorithms and scripts in the computer terminal 118. The software in the computer terminal 118 may be layered with a graphical user interface (GUI) for allowing a user to monitor the toxicity measurement test/

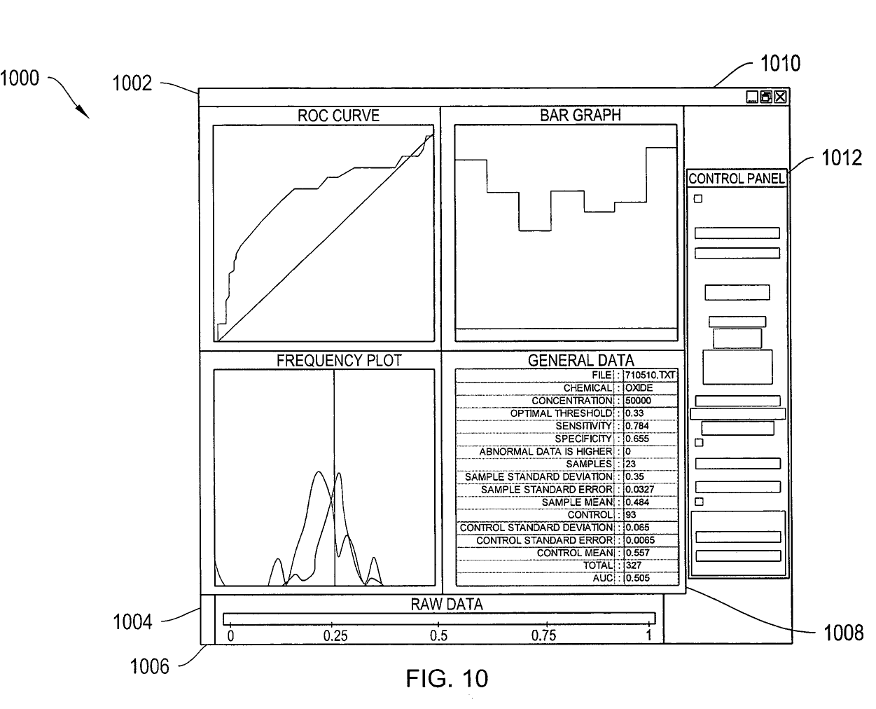

FIG. 10 depicts a graphical user interface (GUI) 1000 according to one illustrative embodiment of the invention. The graphical user interface 1000 includes a receiver operating characteristics (ROC) curve display panel 1002, a frequency plot display panel 1004, a raw data display panel 1006, a general data display panel 1008 and a bar graph display panel 1010. The GUI 1000 allows a user to choose whether the current sample is a continuous sample (e.g., drinking water supply) or a single sample (e.g., dose response assay samples). The toxicity measurement system 102 acquires raw data, calculates the ROC curve, sensitivity and precision of the analysis and then plots of all these characteristics.

Receiver operating characteristics shown in panel 1002 is typically a standard approach to evaluating the sensitivity and specificity of diagnostic procedures such as a toxicity measurement test. Sensitivity, also typically known as true positive fraction, may be the probability of detecting toxic substance when it is actually present. Specificity, true negative fraction, may be the probability of detecting the absence of a toxic substance when it is not present. Typically, a threshold value (cut-off value) is selected and test results are evaluated against this threshold value to determine whether a toxic substance is present or absent. The threshold value used may influence sensitivity and specificity of the method. Typically, a lower threshold value may result in a higher sensitivity and lower specificity. ROC analysis typically estimates a curve which describes the inherent tradeoff between sensitivity and specificity of a diagnostic test. Each point on an ROC curve is associated with a corresponding diagnostic criterion. This point may vary among observers because the diagnostic criteria may vary even when their ROC curves are similar. The ROC curve may be used to determine an optimal threshold for each test. The area under the ROC curve (AUC) shows the average sensitivity over all or substantially all specificities. The AUC may also represent the system's accuracy.

In one embodiment, the NGDR from the tracked path of each organism 106 when compared to a control sample is used to calculate the ROC plot. In certain embodiments, a threshold for discrimination between controls and the sample based on the NGDR is calculated to establish a threat level. In one embodiment, the threat level ranges from about 0-100% based, at least in part, on the area under the ROC curve. In one embodiment, normal conditions exist between 0 and 60%. Yellow alert occurs when the threat level is between 60-70%. A red alert exists when the threat level exceeds 70%. The calculation of a threat level may be accompanied by a sensitivity, specificity, and area under the ROC curve value to allow the user to interpret the results as desired. The data may then be sent to a server for display on the internet. The frequency plot shown in panel 1004 may be based on the mean NGDR for substantially all paths in the organism-sample mixture 110.

Panel 1004 shows the frequency plot including a vertical line representing the optimal threshold value. Panel 1006 displays the calculated data in its raw form (e.g., the NGDR data for both the sample 114 and the control sample such as distilled water sample). Panel 1008 shows some general information about the ROC curve such as chemical, concentration, optimal threshold, sensitivity and specificity at optimal threshold, abnormal data, sample statistics, control sample statistics and AUC. Panel 1012 is a control panel for controlling the display options on the GUI. In one embodiment, the control panel 1012 allows a user to select a desired data series to plot. In such an embodiment, panel 1010 plots a bar graph of the data series selected in the control panel 1012.

The toxicity measurement system 102 may be used to test samples comprising a plurality of toxins with a plurality of organisms 106. Various path characteristics (velocity, direction etc.), statistical calculations (NGDR, threat level etc.) and environmental and test conditions (concentration, temperature, etc.) for one or more toxins as detected using one or more organisms may be stored in the computer terminal 118. A database of toxins, organisms and corresponding characteristics may be built and utilized in identifying toxins.

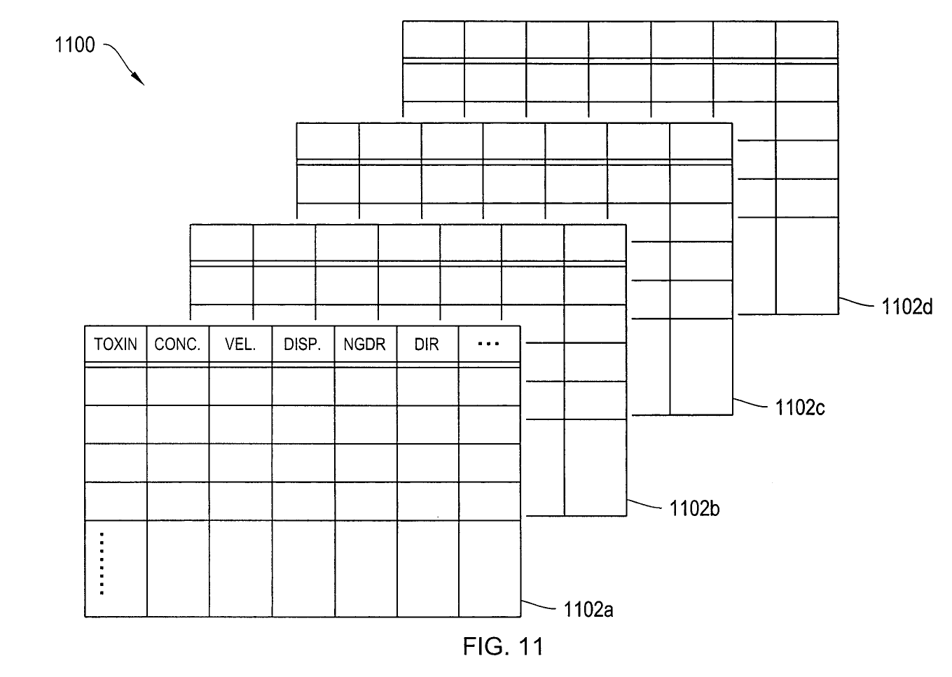

FIG. 11 depicts a scheme for building a database 1100 having organism's 106 responses to various toxins according to one illustrative embodiment of the invention. The database 1100 includes one or more toxicity response matrices 1102a-1102d (generally, “toxicity response matrix 1102”). The toxicity response matrix 1102 includes response characteristics for a particular organism 106 to a plurality of toxins. The response characteristics may include motility responses such as velocity, displacement, NGDR and direction. The response characteristics may also include other response characteristics such as color, shape, physical state and odor. Additionally and optionally, the matrix 1102 may include experimental information such as concentration of toxins in a sample and concentration of organisms needed to produce a response.

The processes described herein may be executed on a conventional data processing platform such as an IBM PC-compatible computer running the Windows operating systems, a SUN workstation running a UNIX operating system or another equivalent personal computer or workstation. Alternatively, the data processing system may comprise a dedicated processing system that includes an embedded programmable data processing unit. For example, the data processing system may comprise a single board computer system that has been integrated into a system for performing micro-array analysis.

The processes described herein may also be realized as a software component operating on a conventional data processing system such as a UNIX workstation. In such an embodiment, the process may be implemented as a computer program written in any of several languages well-known to those of ordinary skill in the art, such as (but not limited to) C, C++, FORTRAN, Java or BASIC. The process may also be executed on commonly available clusters of processors, such as Western Scientific Linux clusters, which are able to allow parallel execution of all or some of the steps in the present process.

As noted above, the order in which the steps of the present method are performed is purely illustrative in nature. In fact, the steps can be performed in any order or in parallel, unless otherwise indicated by the present disclosure. The method of the present invention may be performed in either hardware, software, or any combination thereof, as those terms are currently known in the art. In particular, the present method may be carried out by software, firmware, or microcode operating on a computer or computers of any type. Additionally, software embodying the present invention may comprise computer instructions in any form (e.g., source code, object code, interpreted code, etc.) stored in any computer-readable medium (e.g., ROM, RAM, magnetic media, punched tape or card, compact disc (CD) in any form, DVD, etc.). Furthermore, such software may also be in the form of a computer data signal embodied in a carrier wave, such as that found within the well-known Web pages transferred among devices connected to the Internet. Accordingly, the present invention is not limited to any particular platform, unless specifically stated otherwise in the present disclosure.

Those skilled in the art will know or be able to ascertain using no more than routine experimentation, many equivalents to the embodiments and practices described herein. Accordingly, it will be understood that the invention is not to be limited to the embodiments disclosed herein, but is to be understood from the following claims, which are to be interpreted as broadly as allowed under the law.

What is claimed is:

1. A method of detecting the presence of a toxin in a sample, comprising
providing a chamber including a sample and an organism having a motility response to a toxin in the sample,
acquiring an image showing an organism in the sample at a first instance in time,
measuring based on the image at least one of a centroid, a size of the organism, a shape of the organism and an orientation of the organism,
determining a path characteristic based at least on the image and a similar image from another instance in time, and
detecting the presence of the toxin in the sample based at least on the path characteristic.

2. The method of claim 1, wherein acquiring an image includes obtaining an electronic image using a camera.

3. The method of claim 1, wherein determining a path characteristic includes determining a path of an organism using at least one of a nearest neighbor distance algorithm, voronoi tessellation algorithm and an autocorrelation algorithm.

4. The method of claim 1, wherein the path characteristic includes at least one of speed, acceleration, direction, displacement and rate of change of direction.

5. The method of claim 1, wherein determining a path characteristic includes estimating the path characteristic, based on a mathematical model of the movement of the organism and the captured image.

6. The method of claim 5, wherein the mathematical model comprises dynamical system models including Kalman filter based model.

7. The method of claim 1, further comprising measuring a net to gross displacement ratio of the path characteristic.

8. The method of claim 7, wherein detecting the presence of a toxin includes comparing the net to gross displacement ratio to a threshold.

9. The method of claim 1, wherein the motility response includes at least one of speed, acceleration, direction, displacement and rate of change of direction.

10. The method of claim 1, wherein the organism includes at least one of a single celled organism, multi-celled organism, fresh water organism and salt water organism.

11. The method of claim 1, wherein the organism includes at least one of a ciliate, flagellate and marine algae.

12. The method of claim 11, wherein the ciliate includes at least one of Tetrahymena pyriformis, Tetrahymena malaccensus, Tetrahymena furgisoni, and Glaucoma.

13. The method of claim 11, wherein the flagellate includes at least one of Tetramitus and Bodo.

14. The method of claim 11, wherein the marine algae includes at least one of Chlorophycia, rhodomonas, heterocapsa, dunaliella, and chlamydomonas.

15. The method of claim 1, comprising the step of building a database of toxins, including
providing a set of toxins,
providing at least one organism having a motility response to at least one of the set of toxins
introducing the organism to the at least one of the set of toxins
measuring the motility response of the at least one organism to the at least one of the set of toxins, and
building a database having the at least one of the set of toxins and the corresponding motility response of the at least one organism.

16. The method of claim 1, comprising the step of identifying a toxin in a sample, including
providing a chamber including the sample and an organism having a motility response to a toxin in the sample,
capturing an image of a portion of the chamber having the organism moving in the sample, and
identifying a toxin in the sample by comparing the motility response of the organism to a motility response of an organism in a database of toxins.
